(19) United States
(12) Patent Application Publication
(10) Pub. No.: US 2018/0312791 A1
(43) Pub. Date: Nov. 1, 2018

(54) REACTION DEVICE FOR TINY ORGANISM AND MONITORING DEVICE FOR TINY ORGANISM USING THE SAME

(71) Applicant: Chung Yuan Christian University, Taoyuan City (TW)

(72) Inventors: Yu-Tzu Huang, Taoyuan City (TW); Wen-Yaw Chung, Taoyuan City (TW); Bo-Wei Wu, Taoyuan City (TW); Jyun-Ting Lai, Taoyuan City (TW); Jie-Ru Xu, Taoyuan City (TW)

(21) Appl. No.: 15/832,150

(22) Filed: Dec. 5, 2017

(30) Foreign Application Priority Data
Apr. 28, 2017 (TW) 106114380

(51) Int. Cl. C12M 1/00

(52) U.S. Cl. CPC C12M 23/02; C12M 29/26; C12M 29/24; C12M 29/20

(57) ABSTRACT

A reaction device for a tiny organism includes a chamber, a first support plate, a second support plate and a separation plate. The tiny organism and a culture medium are added in the chamber. The first support plate is disposed on an opening of the chamber. The second support plate is disposed on one side opposite to the side on which the first support plate is disposed, and a third hole and a fourth hole separately correspond to a first hole and a second hole of the first support plate. A first end of the separation plate is located inside the chamber, the first end of the separation plate is spaced from the bottom of the chamber by a predetermined distance, a second end of the separation plate passes through the first support plate to connect to the second support plate.

### CROSS REFERENCE TO RELATED APPLICATION

This application claims the priority benefit of Taiwan Patent Application Serial Number 106114380, filed on Apr. 28, 2017, the full disclosure of which is incorporated herein by reference.

### BACKGROUND

### Technical Field

This present disclosure generally relates to a monitoring device and, more particularly, to a reaction device for a tiny organism and a monitoring device for a tiny organism using the same.

### Related Art

In general, a culture chamber is mostly used to monitor the culture of microorganisms (such as algae) in the water. The microorganism and the culture medium are added in the interior of the culture chamber and a cover plate is covered on the culture chamber. Additionally, a hole is formed on the cover plate, so that gas are inputted through the hole. A detector may be disposed in the hole to be installed and fixed on the cover plate to detect the state of the interior of the culture chamber.

However, bubbles are generated when the gas is inputted into the culture chamber. The bubble may directly contact with the detector, such that the reading data of the detector is not stable, resulting in affecting the readings of the detector. Additionally, the water in the interior of the culture chamber may gradually decrease during the monitoring. Since the detector is fixed on the cover plate, the detector may not contact with the water and may not continue detecting the state of the interior of the culture chamber, thereby decreasing the stability of the monitoring. Therefore, the monitoring manner is required to be improved.

### SUMMARY

The present disclosure provides a reaction device for a tiny organism and a monitoring device for a tiny organism using the same, thereby increasing the fixedness and stability of the detecting unit. Additionally, the detecting unit may effectively continue detecting the state of the interior of the chamber when the water in the interior of the chamber gradually decrease.

The present disclosure provides a reaction device for a tiny organism, which includes a chamber, a first support plate, a second support plate and a separation plate. The tiny organism and a culture medium are added in the chamber. The first support plate is disposed on an opening of the chamber, wherein the first support plate has a first hole and a second hole. The second support plate is disposed on one side opposite to the side on which the first support plate is disposed, wherein the second support plate has a third hole and a fourth hole, a position of the third hole corresponds to a position of the first hole, and a position of the fourth hole corresponds to a position of the second hole. The separation plate has a first end and a second end opposite to the first end, wherein the first end of the separation plate is located inside the chamber, the first end of the separation plate is spaced from the bottom of the chamber by a predetermined distance, the second end of the separation plate passes through the first support plate to connect to the second support plate, and the separation plate is connected to the first support plate.

The present disclosure provides a monitoring device for a tiny organism, which includes a reaction device, a gas pipeline and a detecting electrode. The reaction device includes a chamber, a first support plate, a second support plate and a separation plate. The tiny organism and a culture medium are added in the chamber. The first support plate is disposed on an opening of the chamber, wherein the first support plate has a first hole and a second hole. The second support plate is disposed on one side opposite to the side on which the first support plate is disposed, wherein the second support plate has a third hole and a fourth hole, a position of the third hole corresponds to a position of the first hole, and a position of the fourth hole corresponds to a position of the second hole. The separation plate has a first end and a second end opposite to the first end, wherein the first end of the separation plate is located inside the chamber, the first end of the separation plate is spaced from the bottom of the chamber by a predetermined distance, the second end of the separation plate passes through the first support plate to connect to the second support plate, and the separation plate is connected to the first support plate. The gas pipeline penetrates the first hole and the third hole to enter to an interior of the chamber to provide gas. The detecting electrode penetrates the second hole and the fourth hole to enter to the interior of the chamber, and detects the tiny organism, the culture medium and the gas inside the chamber to generate a detecting signal.

According to the reaction device for the tiny organism and the monitoring device for the tiny organism using the same of the embodiments of the present disclosure, the first support plate covers on the opening of the chamber, the first support plate and the second support plate constitutes a two-layered structure, the first end of the separation plate is disposed in the interior of the chamber, the first end of the separation plate is spaced from the bottom of the chamber by a predetermined distance and the second end of the separation plate passes through the first support plate to connect to the second support plate. Therefore, the detecting unit may be fixed effectively and is more capable of resisting the swirl movement generated by the water in the interior of the chamber due to stirring. When the height of the water surface in the interior of the chamber rises or lowers, the sensing unit may also be raised or lowered for avaoding swaying. When the height of the water surface in the interior of the chamber gradually falls, the sensing unit may effectively contact with the water, such that the detecting unit continues detecting the state of the interior of the chamber and the detecting effect is effectively increased. Additionally, the gas pipeline is separated from the sensing unit by the separation plate such that the bubbles generated by inputting the air to the interior of the chamber may be effectively avoided from directly contacting with the detecting unit to affect the sensing state of the detecting unit, thereby improving the stability of the detecting unit.

It should be understood, however, that this summary may not contain all aspects and embodiments of the present invention, that this summary is not meant to be limiting or restrictive in any manner, and that the invention as disclosed herein will be understood by one of ordinary skill in the art to encompass obvious improvements and modifications thereto.

### BRIEF DESCRIPTION OF THE DRAWINGS

The features of the exemplary embodiments believed to be novel and the elements and/or the steps characteristic of the exemplary embodiments are set forth with particularity in the appended claims. The Figures are for illustration purposes only and are not drawn to scale. The exemplary embodiments, both as to organization and method of operation, may best be understood by reference to the detailed description which follows taken in conjunction with the accompanying drawings in which:

FIG. 1 shows a schematic view of a reaction device for a tiny organism according to an embodiment of the present disclosure;

FIG. 2 shows an exploded view of a reaction device for a tiny organism according to an embodiment of the present disclosure;

FIG. 3 shows a schematic view of a monitoring device for the tiny organism according to an embodiment of the present disclosure;

FIG. 4 shows an exploded view of a part of a monitoring device for the tiny organism according to an embodiment of the present disclosure; and

FIG. 5 shows a circuit diagram of a part of a monitoring device for the tiny organism according to an embodiment of the present disclosure.

### DETAILED DESCRIPTION OF THE EMBODIMENTS

The present disclosure will now be described more fully hereinafter with reference to the accompanying drawings, in which exemplary embodiments of the invention are shown. This present disclosure may, however, be embodied in many different forms and should not be construed as limited to the embodiments set forth herein. Rather, these embodiments are provided so that this present disclosure will be thorough and complete, and will fully convey the scope of the present disclosure to those skilled in the art.

Certain terms are used throughout the description and following claims to refer to particular components. As one skilled in the art will appreciate, manufacturers may refer to a component by different names. This document does not intend to distinguish between components that differ in name but function. In the following description and in the claims, the terms “include/including” and “comprise/comprising” are used in an open-ended fashion, and thus should be interpreted as “including but not limited to”. “Substaintial/substaintially” means, within an acceptable error range, the person skilled in the art may solve the technical problem in a certain error range to achieve the basic technical effect. Additionally the term “couple” or “connect” covers any direct or indirect electrically coupling means. Therefore when one device is electrically connected to another device in the context, that connection may be through a direct electrical connection, or through an indirect electrical connection via other devices and connections. The following description is of the best-contemplated mode of carrying out the invention. This description is made for the purpose of illustration of the general principles of the invention and should not be taken in a limiting sense. The scope of the invention is best determined by reference to the appended claims.

Moreover, the terms “include”, “contain”, and any variation thereof are intended to cover a non-exclusive inclusion. Therefore, a process, method, object, or device that includes a series of elements not only includes these elements, but also includes other elements not specified expressly, or may include inherent elements of the process, method, object, or device. If no more limitations are made, an element limited by “include a/an . . . ” does not exclude other same elements existing in the process, the method, the article, or the device which includes the element.

In the following embodiment, the same reference numerals is used to refer to the same or similar elements throughout.

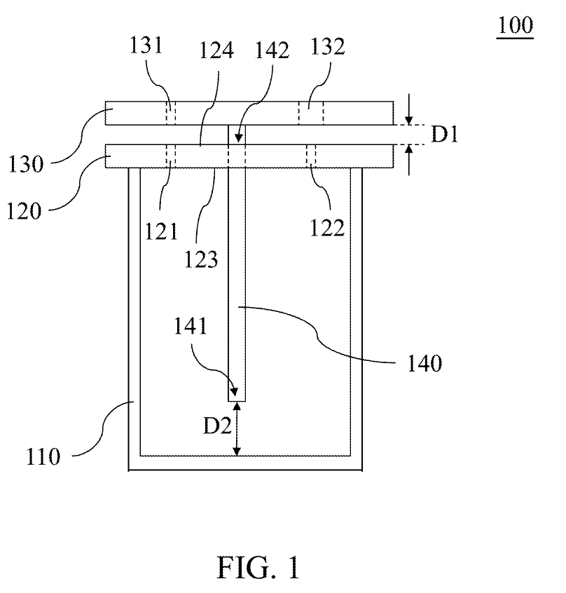

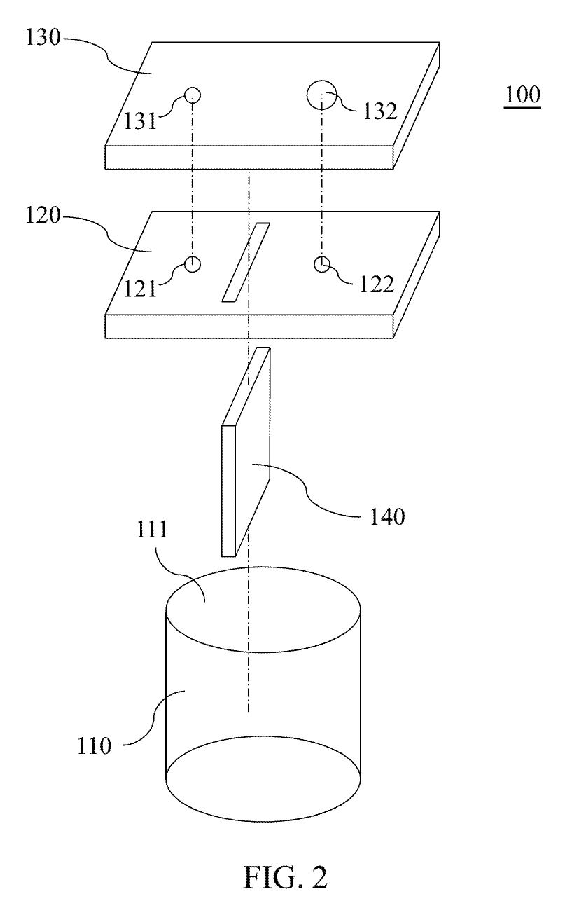

FIG. 1 shows a schematic view of a reaction device for a tiny organism according to an embodiment of the present disclosure. FIG. 2 shows an exploded view of a reaction device for a tiny organism according to an embodiment of the present disclosure. Please refer to FIG. 1 and FIG. 2, a reaction device for a tiny organism 100 includes a chamber 110, a first support plate 120, a second support plate 130 and a separation plate 140. The tiny organism and a culture medium are added in the chamber 110, wherein the tiny organism may include microorganism, such as algae, etc.

The first support plate 120 is disposed on an opening 111 of the chamber 110, i.e. the first support plate 120 may be served as a cover plate of the chamber 100. The first support plate 120 has a first hole 121 and a second hole 122.

The second support plate 130 is disposed on the side 124 opposite to the side 123 on which the first support plate 120 is disposed. In the embodiment, the second support plate 130 and the first support plate 120 are, for example, disposed in parallel, and the second support plate 130 is spaced from the first support plate 120 by a predetermined distance D1. The predetermined distance D1 may be adjusted by the user as required. The second support plate 130 has a third hole 131 and a fourth hole 132. A position of the third hole 131 corresponds to a position of the first hole 121. A position of the fourth hole 132 corresponds to a position of the second hole 122.

The separation plate 140 has a first end 141 and a second end 142 opposite to the first end 141. The first end 141 of the separation plate 140 is located inside the chamber 100. The first end 141 of the separation plate 140 is sapced from the bottom of the chamber 110 by a predetermined distance D2. The second end 142 of the separation plate 140 passes through the first support plate 120 to connect to the second support plate 130, and the separation plate 140 is connected to the first support plate 120.

Further, a shape of the chamber 110 is, for example, cylindrical, and the material of the chamber 110 may be a glass with high light transmission, such that the light source may be averagely distributed, and the tiny organism may absorb the light source more easily and is not be stuck in the gap. The shape of the above chamber 110 being cylindrical is one implementation of the embodiment, and not limited to the present disclosure. The shape of the chamber 110 may be adjusted by the user as required, such as square or rectangle.

Additionally, the materials of the first support plate 120, the second support plate 130 and the separation plate 140 may be transparent material, such as acrylic, etc. Moreover, in the embodiment, the first hole 121 and the third hole 131 is suitable to allow that a gas pipeline passes through to enter the interior of the chamber 110, such that the gas may be inputted to the interior of the chamber 110. The second hole 122 and the fourth hole 132 is suitable to allow that a terminal of a detecting unit passes through to enter the interior of the chamber 110, such that a state of the interior of the chamber 100 may be detected.

In the embodiment, the diameter of the fourth hole 132 and the diameter of the second hole 122 may be adjusted according to the appearance of the sensing unit. For example, the diameter of the fourth hole 132 may be set as greater than the diameter of the second hole 122. Therefore, two-layer holding structure are formed by the first support plate 120 and the second support plate 130, such that the detecting unit 330 may be fixed effectively and is more capable of resisting the swirl movement generated by the water in the interior of the chamber 110 due to stirring.

When the height of the water surface in the interior of the chamber 110 rises or falls, the sensing unit may also be raised or lowered and the sensing unit does not also be shook. When the height of the water surface in the interior of the chamber 110 gradually falls, the sensing unit may effectively contact with the water, such that the detecting unit continues detecting the state of the interior of the chamber 100 and the detecting effect is effectively increased. Additionally, the gas pipeline is separated from the sensing unit through the separation plate 140, it may effectively avoid the bubbles generated by inputting the air to the interior of the chamber 110 directly from contacting with the detecting unit and from affecting the sensing state of the detecting unit, thereby improving the stability of the detecting unit.

Furthermore, in the above embodiment, the amount of the fourth hole 132 and the amount of the second hole 122 are taken only one for example, but not limited to the present disclosure. The amount thereof may be adjusted by the user as required, and the amount of the fourth hole 132 and the amount of the second hole 122 may correspond to the amount of the sensing unit. For example, when the amount of the sensing unit is two, the amount of the fourth hole 132 and the amount of the second hole 122 are also two. When the amount of the sensing unit is three, the amount of the fourth hole 132 and the amount of the second hole 122 are also three. The rest is analogy.

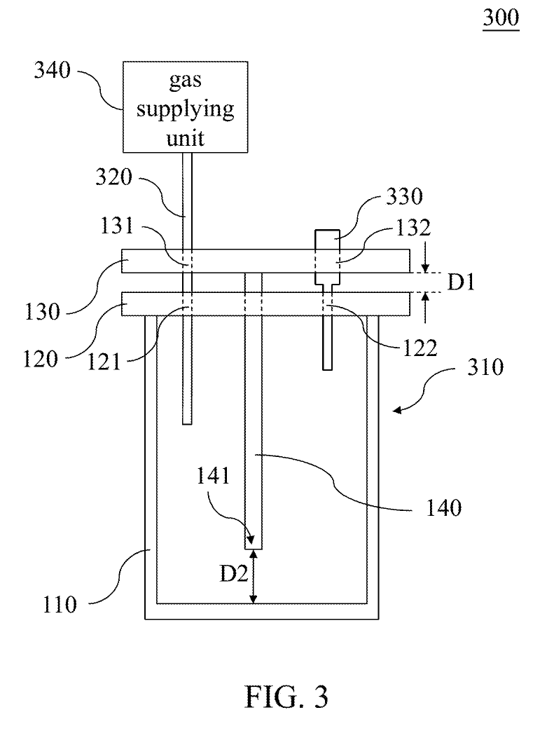

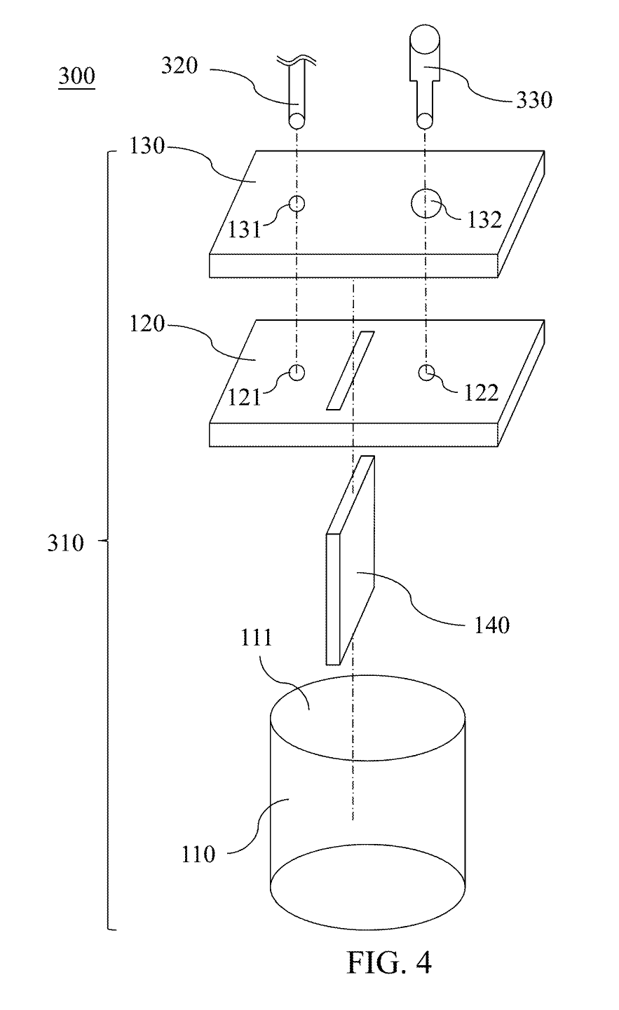

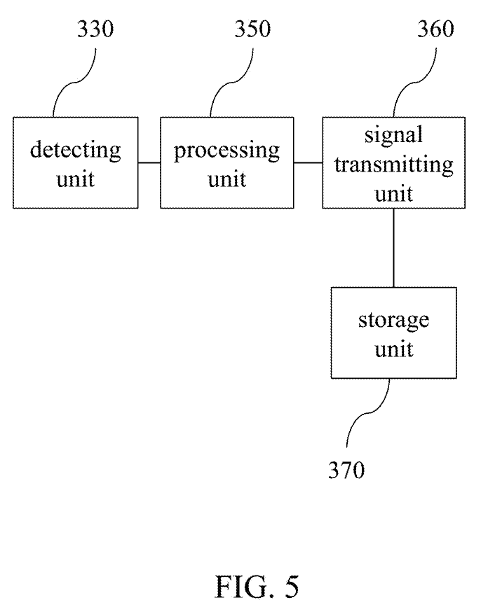

FIG. 3 shows a schematic view of a monitoring device for the tiny organism according to an embodiment of the present disclosure. FIG. 4 shows an exploded view of a part of a monitoring device for the tiny organism according to an embodiment of the present disclosure. FIG. 5 shows a circuit diagram of a part of a monitoring device for the tiny organism according to an embodiment of the present disclosure. Please refer to FIG. 3, FIG. 4 and FIG. 5, a monitoring device for the tiny organism 300 includes a reaction device 310, a gas pipeline 320 and a detecting electrode 330

In the embodiment, the reaction device 310 includes a chamber 110, a first support plate 120, a second support plate 130 and a separation plate 140. The disposing relationship of the reaction device 310, the chamber 110, the first support plate 120, the second support plate 130 and the separation plate 140 may refer to the description of the embodiments of FIG. 1 and FIG. 2, and the description thereof is omitted.

The gas pipeline 320 passes through the first hole 121 and the third hole 131 to enter to an interior of the chamber, so as to provide a gas. Furthermore, the monitoring device for the tiny organism 300 may further includes a gas supplying unit 340. The gas supplying unit 340 is connected to the gas pipeline 320, such that the gas supplied by the gas supplying unit 340 is inputted to the interior of the chamber 110 through the gas pipeline 320.

The detecting electrode 330 passes through the second hole 122 and the fourth hole 132 to enter to the interior of the chamber 110, and detects the tiny organism, the culture medium and the gas inside the chamber, so as to generate a detecting signal. In the embodiment, the detecting signal may include, for example, pH value, dissolved oxygen, temperature, conductivity and/or brightness.

In the embodiment, the diameter of the fourth hole 132 and the diameter of the second hole 122 may be adjusted according to the appearance of the sensing unit 330. For example, the diameter of the fourth hole 132 may be set as greater than the diameter of the second hole 122. Therefore, two-layer holding structure are formed by the first support plate 120 and the second support plate 130, such that the detecting unit 330 may be fixed effectively, and is more capable of resisting the swirl movement generated by the water in the interior of the chamber 110 due to stirring.

When the height of the water surface in the interior of the chamber 110 rises or falls, the sensing unit 330 may also be raised or lowered and the sensing unit may be avoided from swaying. When the height of the water surface in the interior of the chamber 110 gradually falls, the sensing unit 330 may effectively contact with the water, such that the detecting unit continues detecting the state of the interior of the chamber 100 and the detecting effect is effectively increased. Additionally, the gas pipeline 320 is separated from the sensing unit 330 through the separation plate 140, it may effectively avoid the bubbles generated by inputting the air to the interior of the chamber 110 from directly contacting with the detecting unit 330 and from affecting the sensing state of the detecting unit 330, thereby improving the stability of the detecting unit 330.

Furthermore, in the above embodiment, the amount of the fourth hole 132 and the amount of the second hole 122 are taken only one for example, but not limited to the present disclosure. The amount thereof may be adjusted by the user as required, and the amount of the fourth hole 132 and the amount of the second hole 122 may be corresponded to the amount of the sensing unit 330. For example, when the amount of the sensing unit 330 is two, the amount of the fourth hole 132 and the amount of the second hole 122 are also two. When the amount of the sensing unit 330 is three, the amount of the fourth hole 132 and the amount of the second hole 122 are also three. The rest is analogy.

Further, the monitoring device for the tiny organism 300 further includes a processing unit 350, a signal transmitting unit 360 and a storage unit 370. The processing unit 350 is connected to the detecting electrode 330 and receives the detecting signal, so as to generate a processing signal. The signal transmitting unit 360 is connected to the processing unit and receives and transmits the processing signal. In the embodiment, the signal transmitting unit 360 may be a wired or wireless transmitting unit, so as to transmit the processing signal through the wired or wireless manner. The storage unit 370 is connected to the signal transmitting unit 350 and receives and storing the processing signal. Therefore, the user may read out the information stored in the storage unit 370, so as to know the growth state of the tiny organism in interior of the chamber 110.

As mentioned above, According to the reaction device for the tiny organism and the monitoring device for the tiny organism using the same of the embodiments of the present disclosure, the first support plate covers on the opening of the chamber, the first support plate and the second support plate constitutes a two-layered structure, the first end of the separation plate is disposed in the interior of the chamber, the first end of the separation plate is spaced from the bottom of the chamber by a predetermined distance and the second end of the separation plate passes through the first support plate to connect to the second support plate. Therefore, the detecting unit may be fixed effectively and is more capable of resisting the swirl movement generated by the water in the interior of the chamber due to stirring. When the height of the water surface in the interior of the chamber rises or lowers, the sensing unit may also be raised or lowered and the sensing unit may be avoided from swaying. When the height of the water surface in the interior of the chamber gradually falls, the sensing unit may effectively contact with the water, such that the detecting unit continues detecting the state of the interior of the chamber and the detecting effect is effectively increased. Additionally, the gas pipeline is separated from the sensing unit by the separation plate, it may effectively avoid the bubbles generated by inputting the air to the interior of the chamber from directly contacting with the detecting unit and from affecting the sensing state of the detecting unit, thereby improving the stability of the detecting unit.

Although the present disclosure has been explained in relation to its preferred embodiment, it does not intend to limit the present disclosure. It will be apparent to those skilled in the art having regard to this present disclosure that other modifications of the exemplary embodiments beyond those embodiments specifically described here may be made without departing from the spirit of the invention. Accordingly, such modifications are considered within the scope of the invention as limited solely by the appended claims.

What is claimed is:

1. A reaction device for a tiny organism, comprising:
a chamber, wherein a tiny organism and a culture medium are added in the chamber;
a first support plate, disposed on an opening of the chamber, wherein the first support plate has a first hole and a second hole;
a second support plate, disposed on one side opposite to the side on which the first support plate is disposed, wherein the second support plate has a third hole and a fourth hole, a position of the third hole corresponds to a position of the first hole, and a position of the fourth hole corresponds to a position of the second hole; and
a separation plate, having a first end and a second end opposite to the first end, wherein the first end of the separation plate is located inside the chamber, the first end of the separation plate is spaced from the bottom of the chamber by a predetermined distance, the second end of the separation plate passes through the first support plate to connect to the second support plate, and the separation plate is connected to the first support plate.

2. The reaction device for the tiny organism as claimed in claim 1, wherein a shape of the chamber is cylindrical.

3. The reaction device for the tiny organism as claimed in claim 1, wherein a diameter of the fourth hole is greater than a diameter of the second hole.

4. A monitoring device for a tiny organism, comprising:
a reaction device, comprising:
a chamber, wherein a tiny organism and a culture medium are added in the chamber;
a first support plate, disposed on an opening of the chamber, wherein the first support plate has a first hole and a second hole;
a second support plate, disposed on one side opposite to the side on which the first support plate is disposed, wherein the second support plate has a third hole and a fourth hole, a position of the third hole corresponds to a position of the first hole, and a position of the fourth hole corresponds to a position of the second hole; and
a separation plate, having a first end and a second end opposite to the first end, wherein the first end of the separation plate is located inside the chamber, the first end of the separation plate is spaced from the bottom of the chamber by a predetermined distance, the second end of the separation plate passes through the first support plate to connect to the second support plate, and the separation plate is connected to the first support plate
a gas pipeline, passing through the first hole and the third hole to enter to an interior of the chamber, so as to provide a gas; and
a detecting electrode, passing through the second hole and the fourth hole to enter to the interior of the chamber, and detecting the tiny organism, the culture medium and the gas inside the chamber, so as to generate a detecting signal.

5. The monitoring device for the tiny organism as claimed in claim 4, wherein the detecting signal comprises pH value, dissolved oxygen, temperature, conductivity and/or brightness.

6. The monitoring device for the tiny organism as claimed in claim 4, further comprising:
a processing unit, connected to the detecting electrode and receiving the detecting signal, so as to generate a processing signal;
a signal transmitting unit, connected to the processing unit, and receiving and transmitting the processing signal; and
a storage unit, connected to the signal transmitting unit, and receiving and storing the processing signal.

7. The monitoring device for the tiny organism as claimed in claim 4, wherein a shape of the chamber is cylindrical.

8. The monitoring device for the tiny organism as claimed in claim 4, wherein a diameter of the fourth hole is greater than a diameter of the second hole.

9. The monitoring device for the tiny organism as claimed in claim 4, further comprising:
a gas supplying unit, connected to the gas pipeline and supplying the gas.
